(19) United States
(12) Patent Application Publication
(10) Pub. No.: US 2008/0130827 A1
(43) Pub. Date: Jun. 5, 2008

(54) Detection device for detecting an object by x-ray radiation in different detecting directions

(75) Inventor: Klaus Klingenbeck-Regn, Nurnberg (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/974,881

(22) Filed: Oct. 16, 2007

(30) Foreign Application Priority Data
Oct. 20, 2006 (DE) 10 2006 049 575.6

(51) Int. Cl. A61B 6/03

(52) U.S. Cl. 378/8; 378/4

(57) ABSTRACT

A detection device for detection of an object in up to three dimensions by means of x-ray radiation in directions of detection differing from each other.

The invention relates to a detection device for detecting an object in up to three dimensions with an x-ray source and a detector for the x-rays arranged in a detection plane, which is arranged and embodied so as to detect the x-rays and to create at least one 2D dataset which represents the object in a projection through the object onto the detection plane. The detection device 1 also features a C-arm connected to the x-ray source and to the detector and a control facility effectively linked to the C-arm, which is embodied to move the C-arm, depending on a control signal received on the input side, in at least two or three rotational degrees of freedom and to hold it in a detection position represented by the control signal. The detection device is embodied to create a 3D data set which represents the object in three dimensions, especially in a overhead view or a section, and to output this together with the at least one 2D dataset for reproduction by means of at least one image display unit. The detection device features a position memory for position datasets, which each represent a detection position and, depending on a user interaction signal, can read out at least one position dataset from the position memory and create a control signal corresponding to the position dataset representing a detection position for moving the C-arm into the detection position and create a 2D dataset there by means of the detector.

A detection device for detection of an object in up to three dimensions by means of x-ray radiation from different directions of detection.

The invention relates to a detection device for detecting an object in up to three dimensions. The detection device features an x-ray source which is embodied for emitting x-ray radiation. The detection device also features a detector for the x-rays arranged in a detection plane, which is arranged and embodied so as to detect the x-rays and to create at least one 2D dataset which represents the object in a projection through the object onto the detection plane. The detection device is embodied to create a 3D dataset representing the object in three spatial dimensions, especially by means of back projection from a plurality of 2D datasets which represent the object from different directions of detection in a projection through the object and to keep the 3D dataset stored in a memory. The detection device also features a C-arm, which is connected to the x-ray source and to the detector. The detector device features a control facility effectively connected to the C-arm which is embodied to move the C-arm as a function of a control signal received on the input side in at least two or three rotational degrees of freedom and to hold it in a detection position represented by the control signal. The detection device is embodied to create an image dataset from the 3D dataset which represents the object, especially a view onto the object, a view through the object or a section through the object, and to output this together with the at least one 2D dataset for reproduction by means of at least one image display unit.

The underlying object of the invention is to specify a detection device for which the ease of operation is improved.

This object is achieved by a detection device of the type mentioned at the start. The detection device has a position memory for position datasets which each represent a detection position. The detection device is embodied, depending on a user interaction signal, to read out at least one position dataset from the position memory and to create a control signal; corresponding to the position dataset representing a detection position for moving the C-arm into the recording position and to create a 2D dataset there by means of the detector.

Through the inventive detection device a user of the detection device can advantageously move to a predetermined sequence of detection positions provided for an intervention and create at least one 2D dataset in the detection position in each case. The user can advantageously observe the object represented by the 2D dataset on an image display unit together with the 3D dataset and thus follow the intervention—in-vivo—by means of the at least one 2D dataset and simultaneously observe a view from above, a view through or a section through the object represented by the 3D dataset. The detection device, to store the 3D dataset in a memory, can feature a connection for a memory, especially a data bus, or the memory itself.

In a preferred embodiment the detection device features a local sensor which is embodied to detect a location of a medical instrument in a spatial area provided for detection of the object and to create an instrument dataset representing the instrument location and assign this to an area of the 3D dataset corresponding to the instrument location. The detection device is embodied to create the image dataset depending on the instrument dataset in such a way that the image dataset additionally represents the medical instrument.

The location sensor enables a user to observe a position of a medical instrument during an intervention within an object represented by the 3D dataset displayed by an image display.

In an advantageous embodiment the location sensor is an electromagnetic location sensor which is embodied for detecting an instrument location by means of at least two, preferably three electromagnetic fields aligned differently from each other end to create an instrument dataset which represents the instrument location.

In another embodiment the location sensor is an ultrasound location sensor which is embodied, by means of two ultrasound sensors connected to the instrument at a distance from each other and three ultrasound receivers at a distance from the ultrasound sensors within a space, for example electret condenser microphones, depending on a delay time difference, to detect from ultrasound signals created by the ultrasound generator a spatial instrument location of the medical instrument and to create a corresponding instrument dataset which represents the instrument location.

In another embodiment the location sensor is embodied to detect a spatial orientation of a magnetizable or of a permanent magnetic object, especially from two, preferably from three different detection directions, and depending on the spatial orientation of the magnetizable or permanent magnetic object, to detect a spatial location of the magnetizable or permanent magnetic object. The magnetizable or permanent magnetic object can for example be connected to the medical instrument, especially in the area of a catheter end or in the area of an end of a guide wire or of another medical or surgical instrument. The location sensor is embodied in this embodiment for creating an instrument dataset which represents the location of the magnetizable or permanent magnetic object.

In another embodiment the location sensor is an optical location sensor which, by means of electromagnetic rays, especially in the infrared wavelength range, can detect a location of the instrument, especially interferometrically and can create an instrument dataset representing an instrument location.

In a preferred embodiment the detection device features a coordinate memory and is embodied to create and object coordinate dataset representing at least one detection location of the 2D dataset and to store the object coordinate dataset in the coordinate memory. The detection device is further embodied to read out the object coordinate dataset, stored in this coordinate memory and output the instrument location in relation to the object coordinate dataset read out or in the form of object coordinates.

In a preferred embodiment the detection device features an image processing unit which is embodied to create a 2D dataset from the 3D dataset which represents a projection through the object represented by the 3D dataset, especially on a virtual detection plane, and to output this on the output side. A virtual projection result can advantageously be created from any given direction of detection by the image processing unit, which is not possible for example by means of a second detector and a second x-ray source, which can be connected by means of a C-arm. In an advantageous embodiment the image processing unit can create the 3D dataset from the 2D datasets.

In an advantageous embodiment the detection device is embodied to create a chronological sequence of 2D datasets by means of a detector, which each represent detection results of the objects which follow each other in time. This allows a user of the detection device advantageously to observe an object provided for an intervention in-vivo.

In a preferred embodiment the detection device, especially the image processing unit, is embodied to subtract at least two 2D datasets for corresponding detection locations from each other. Advantageously, in a fluoroscopic detection of an object, this allows an area highlighted by means of an x-ray contrast medium, for example a vessel system, especially of a heart to be extracted or an image contrast to be improved. To this end the detection can be further embodied to create an angio 2D dataset which represents a subtraction result of the subtraction.

In a preferred embodiment the detection device features a movement sensor which is embodied to detect an object movement of the object and to create a movement signal which represents the object movement. The detection device is also embodied, depending on the movement signal, to create a 3D dataset or to create a 2D dataset from the 3D dataset. The movement sensor can for example be an acceleration sensor embodied as able to be connected to the object or an interferometric optical movement sensor, which can detect the movement of the object without contact. An object movement can advantageously be detected by the movement sensor and, depending on the object movement a 2D dataset corresponding to an object movement, can be created from the 3D dataset representing a view onto, a view through or a section through the object represented by the 3D dataset and reproduced once again by means of the image display unit.

In an advantageous embodiment the detection device can feature a correlation unit for this purpose which is embodied, depending on a similarity parameter, especially by means of cross correlation, to determine from the 3D dataset a view onto, through or around the object corresponding to the object movements and to create a corresponding image dataset. In a preferred embodiment of the detection device the control facility is embodied to move the C-arm depending on the control signal in at least two or three translational degrees of freedom and to keep it held in a detection position represented by the control signal. This advantageously allows a patient table to remain fixed in a position provided for an intervention, so that a user does not have to move around during an intervention.

The invention also relates to a method for detecting an object in up to three dimensions by means of x-rays, in which a plurality of 2D datasets is created by means of a detector for the x-rays, with the 2D datasets representing the object from different directions of detection in each case in a projection through the object and a 3D dataset representing the object in three spatial dimensions is created from the 2D datasets, which represents the object in three dimensions. In the method an image dataset which represents the object, especially a view onto the object, a view through the object or a section through the object is created from the 3D dataset and this, together with at least one 2D dataset or chronological sequence of 2D datasets created by the detector, especially in-vivo, representing the object in a projection through the object is reproduced by means of an image display unit. Furthermore at least two position datasets are kept stored, which respectively represent different detection positions in relation to each other for creating a 2D dataset and for the position datasets—especially for a part of the position datasets or for each position dataset—a detection of the object is undertaken depending on a user interaction signal corresponding to the position represented by the position datasets in each case. Furthermore the 2D dataset or the chronological sequence of 2D datasets is created in the detection position by means of the detector, especially in-vivo. The method advantageously enables a user, especially a doctor, to detect an object in three dimensions during an intervention, to create a corresponding 3D detection result, and during a following intervention to create different 2D detection results in relation to each other fluoroscopically, namely in-vivo, and to observe these together with the 3D detection results on an image display unit.

In an advantageous embodiment of the method a subtraction result can be created from at least two 2D datasets by means of subtraction for corresponding detection locations and an angio 2D dataset representing this result can be created and this can be reproduced together with the image dataset by means of the image display unit.

In this way a user, by enriching a contrast means for example, can create a detection result which represents a vessel tree of the object.

In an advantageous embodiment of the method a location of a medical instrument is detected in spatial area provided for the detection of the object and an instrument dataset representing the instrument is created and assigned to an area of the 3D dataset corresponding to the instrument location, and from the 3D dataset an image dataset is created which represents the object, especially a view onto the object a view through the object or a section through the object together with the instrument.

This allows a user to observe a medical instrument, for example a catheter, especially an ablation catheter, a guide wire or a high-frequency surgical instrument, together with the 3D detection result, with the medical instrument having been detected as well in the 3D detection results detected in-vivo and is thus also represented in the 2D detection result.

The invention will now be described below with reference to Figures and further exemplary embodiments.

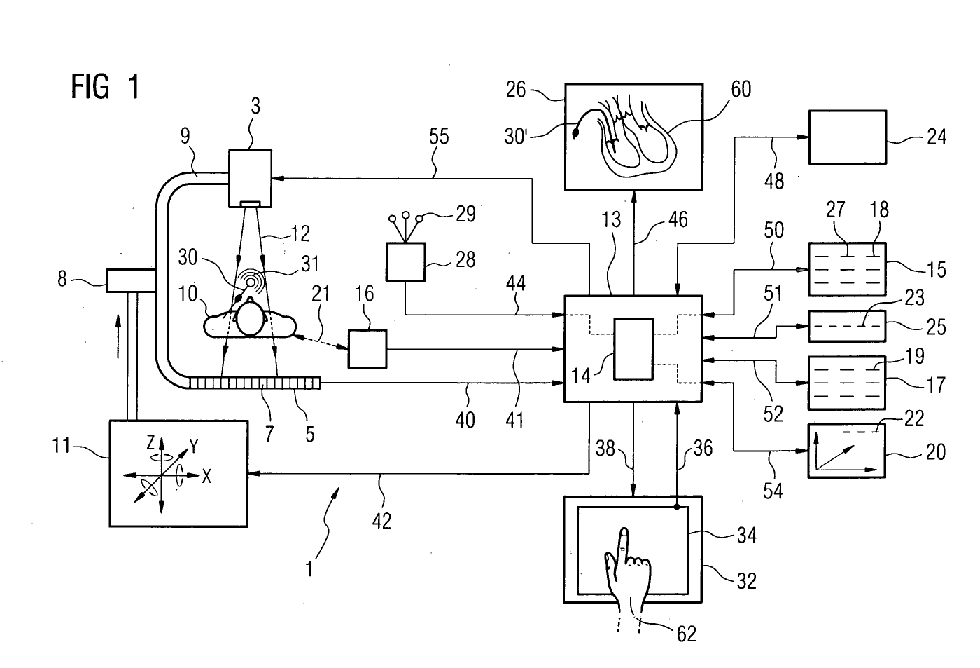

FIG. 1 shows a schematic diagram of an exemplary embodiment for a detection device for detecting an object by means of x-rays with an x-ray source and a detector;

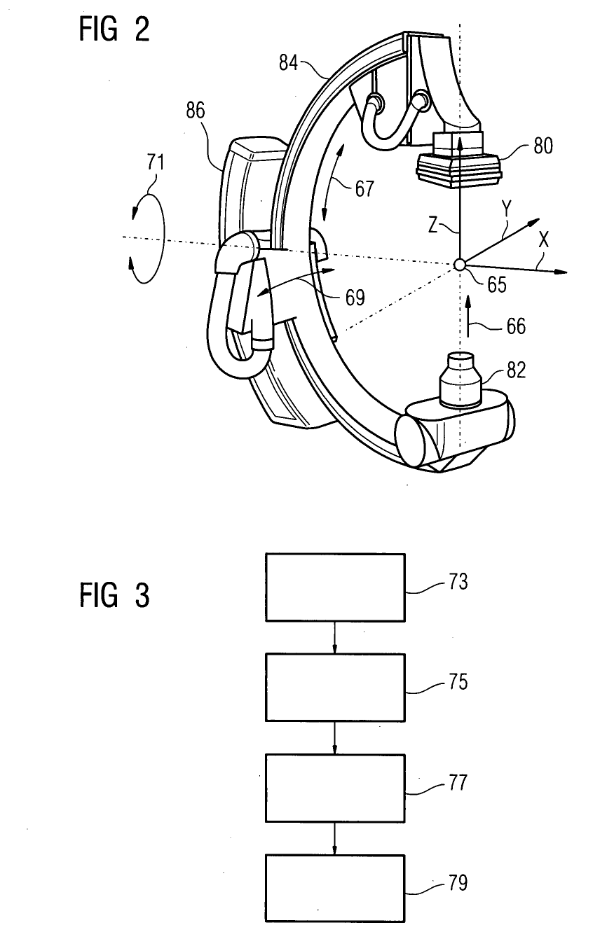

FIG. 2 shows a schematic diagram of an exemplary embodiment for a C-arm;

FIG. 3 shows an exemplary embodiment for a method for recording an object by means of x-rays.

FIG. 1 shows a schematic diagram of an exemplary embodiment for a detection device 1 with an x-ray source 3 and a detector 5. The detector 5 features a plurality of detector matrix elements, of which the detector matrix element 7 is shown as a typical example. The x-ray source 3 is connected to the detector 5 by means of a C-arm 9 such that an object 10 can be detected by means of x-rays 12 emitted by the x-ray source 3 in a projection through the object 10 onto the detector 5. The C-arm 9 is supported to allow it to pivot and can be pivoted in three rotational degrees of freedom, especially around an axis X, an axis Y or an axis Z. The axes X, Y and Z together form an orthogonal system. The C-arm can also be moved in three translational degrees of freedom, especially in parallel to the axis X, in parallel to the axis Y or in parallel to the axis Z. To this end the C-arm 9 is connected by means of an adjustment mechanism 8 to a control facility 11 such that the C-arm 9 can be moved rotationally and/or translationally. To this end the control facility is embodied to move the C-arm 9 by means of the control mechanism 8 depending on a control signal received on the input side and to hold it in a detection position represented by the control signal.

The detector matrix elements of the detector 5 are embodied in each case to receive x-ray radiation and depending on the received x-ray radiation, to create a detector matrix element signal which represents a ray intensity of the received x-ray. The detector matrix elements can feature selenium of silicon, especially amorphous silicon. The detection device 1 also features a central processing unit 13. The central processing unit 13 features an assignment unit 14. The detection device 1 also features a memory 15 and a memory 17. The memory 15 is embodied to store 2D datasets, of which the 2D dataset 18 is shown as an example. The memory 17 is embodied to store at least one 3D dataset, of which the 3D dataset 19 is shown as an example.

The detection device 1 also features a position memory 25, which is embodied to store position datasets which each represent a respective detection position. The position dataset 23 is identified as an example.

The detection device 1 also features a coordinate memory 20, which is embodied to store an object coordinate dataset, with the object coordinate dataset 22 being identified as an example. The memory 15, the memory 17 and the memory 20 can be implemented together by a common memory. The memories 15, 17, 20 and 25 are embodied in each case as read-write memories, especially as non-volatile read-write memories.

The detection device 1 also features an image processing unit 24. The image processing unit 24 is embodied, from a plurality of 2D datasets which each represent a detection result of a projection of x-ray radiation 12 through the object 10 from directions of detection which differ from each other in each case—to this end for example the x-ray source 3 together with the detector 5 and the C-arm 9 can have been pivoted around the object 10 by the control device 11—to create a 3D dataset which represents the object 10 in three dimensions. The 3D dataset can for example be created by means of back projection, especially filtered back projection by the image processing unit 24. The 3D dataset can represent a plurality of voxel object points which together represent the object 10 in three dimensions.

The detection device 1 also features an image display unit 26. The detection device 1 also features an input unit 32 with a touch-sensitive surface 34. The input unit 32 in this embodiment features an image display unit with the touch-sensitive surface 34. The touch-sensitive surface 34 is embodied, as a function of being touched—by a user's hand 62—to create a user interaction signal which represents the location at which the touch-sensitive surface 34 was touched and to output this on the output side. The detection device 1 also features a location sensor 28. The location sensor 28 features at least one antenna 29, which is embodied to detect an electromagnetic field 31 of the medical instrument 30. The medical instrument 30 is embodied to create the electromagnetic field 31. The location sensor 28 is embodied, depending on the detected electromagnetic field 31, to create an instrument dataset which represents the location of the instrument 30 and to output this on the output side. The touch-sensitive surface 34 is connected on the output side via a connecting line 36 to the central processing unit 13. The central processing unit 13 is connected via a connecting line 38 to the input unit 32 and is connected there to the image display unit of the input unit 32. The detector 5 is connected on the output side via a connecting line 40 to the central processing unit 13. The central processing unit 13 is connected on the output side via a connecting line 42 to the pivot device 11. The central processing unit 13 is connected on the input side via a connecting line 44 to the location sensor 28, via a connecting line 46 to the image display unit 26, via a connecting line 48 to the image processing unit 24, via a connecting line 50 to the memory unit 15, via a connecting line 52 to the memory unit 17 and via a connecting line 54 to the coordinate memory 20. The detection device 1 also features a movement sensor 16 which can detect by means of an optical beam 21—for example an electromagnetic beam in the infrared wavelength range—an object movement especially interferometrically, and can create a movement signal representing the object movement. The movement sensor 16 is connected on the output side via a connecting line 41 to the central processing unit 13. The connecting lines 48, 50, 51, 52 or 54 can be embodied bidirectionally in each case and can each be a data bus.

The functions of the detection device 1 will now be explained below:

The central processing unit 13 is embodied, depending on a user interaction signal received on the input side via the connecting line 36, to output a control signal for creating the x-ray beam 12 by means of the x-ray source 3 and to output this signal on the output side via the connecting line 55. The control signal for creating the x-ray beam 12 can for example represent an acceleration voltage, a radiation time or a quantity of electrical charge generating the x-rays 12. The detector 5 can detect the x-rays 12 created by the x-ray source 3 through the object 10 in a projection onto a detection plane in which the detector 5 is arranged and create a 2D dataset which represents the object 10 in a projection through the object 10 onto the detection plane. The 2D dataset in this case represents a 2D matrix, formed from matrix elements, each of which represents an intensity value which matches the correspondingly assigned detector matrix element signal of a detector matrix element. The central processing unit 13 can receive the 2D dataset via the connecting line 40 on the input side and store it via the connecting line 50 in the memory 15. The 2D dataset 18 is identified as an example in this memory.

The central processing unit 13 can, to create further 2D datasets which represent the object 10 recorded from different directions of detection—for example depending on a user interaction signal received via the connecting line 36—read out from the position memory 25 at least one position dataset and create a control signal corresponding to the position dataset, representing a detection position, and send this on the output side via the connecting line 42 to the control facility 11. The control facility 11 can, depending on the control signal, move the C-arm 9 together with the detector 5 and the x-ray source 3, around the object 10—in accordance with the three rotational and the three translational degrees of freedom—into the position corresponding to the control signal and fix it there.

In a further intervention process the C-arm 9 can be moved in accordance with a further position dataset into a further detection position as previously described. The central processing unit 13 can then send a further signal to create an x-ray 12 via the connecting line 55 to the x-ray source 3 and receive a detection result created by the detector 5, namely at least on 2D-dataset, via the connecting line 40 and store it via the connecting line 50 in the memory 15. The central processing unit 13 can in this way create a plurality of 2D datasets, which each represent the object 10 in a projection through the object onto a detection plane recorded from different directions of detection in each case. The central processing unit 13 can now—for example depending on a user interaction signal received via the connecting line 36—read out the 2D datasets from memory 15 via the connecting line 50 and send them via connecting line 48 to the image processing unit 24.

The image processing unit 24 can create a 3D dataset from the received 2D datasets, for example by means of a back projection algorithm, especially a filtering back projection algorithm. The image processing unit 24 can send back the 3D dataset which represents the object 10 in three dimensions via the connecting line 48 to the central processing unit 13. The 3D dataset can represent a plurality of voxel object points, each of which represents a value of an absorption coefficient for x-rays at an object location and thus together represent the object 10 in three dimensions. The central processing unit 13 can store the 3D dataset received via the connecting line 48 in the memory 17 via the connecting line 52. The 3D dataset 19 is identified as an example in this memory. The central processing unit 13 can receive an instrument dataset which represents an instrument location of the instrument 30 on the input side via the connecting line 44. The instrument 30 is arranged in this exemplary embodiment within the object 10. The central processing unit 13 can for example, for calibration of the detection device 1, receive via the connecting line 44 an instrument dataset and create at least one object coordinate dataset representing the detection location of the 3D dataset and send this via the connecting line 44 to the coordinate memory 20 and store it there. The object coordinate dataset 22 is identified as an example and represents either at least two detection locations, each for a voxel of the 3D dataset, or a detection location for a voxel and a spatial orientation, for example in the form of a vector, which represents an orientation of the 3D dataset.

Unlike the procedure described above, before a read-out of the position memory 25, a 3D dataset can be created from a plurality of 2D datasets and stored in the memory 17. Subsequently a user—using their hand 62 for example—creates a user interaction signal for reading out a position dataset from the position memory 25 and moving to a further detection position.

The central processing unit 13 can for example create a user menu signal and send this via the connecting line 38 to the input unit 32. The user menu signal can represent the detection positions kept stored in the memory 25, especially alpha-numerically or in the form of graphic symbols. The user can create a user interaction signal corresponding to a detection position and send this via the connecting line 36 to the central processing unit 13. The central processing unit can create a control signal for the corresponding detection position and send this to the control facility 11 and can create a 2D dataset by means of the x-ray source 3 and the detector 5 and store it in memory 15.

The detection device 1 can for example create at a detection position—in-vivo—a fluoroscopic 2D dataset or a chronological sequence of 2D datasets. The detection device can for example create by means of the image processing unit 24 an angio 2D dataset which represents a vessel system of the detected object 10. To this end the image processing unit 24 can subtract at least two 2D datasets from each other for each detection location, especially for each matrix element of a matrix represented by the 2D dataset—and create the angio 2D dataset as a subtraction result. The angio 2D dataset 27 is identified as an example. Thus the detection device can increase an image contrast created by means of a contrast medium.

During an intervention the central processing unit, especially an assignment unit 14 can assign an instrument dataset received via the connecting line 44 to an objected location represented by a part of the 3D dataset and create an assignment result which corresponds to the instrument location within the space represented by the 3D dataset. The central processing unit 13 can, for example by means of the assignment result created by the assignment unit 14, create an image dataset which represents the object 10, especially for example a heart 60 of the object 10 in three dimensions together with the instrument 30.

The central processing unit 13 can for example also create a 3D dataset from angio 2D datasets, so that the 3D dataset represents a vessel system of the object.

The central processing unit can, during a further intervention process, create a chronological sequence of 2D datasets or angio 2D datasets and receive these via the connecting line 40, keep them stored in the memory 15, and read these out again for joint reproduction with the image dataset by means of the image display unit 26. The image display unit 26 typically reproduces the heart 60 and the instrument 30′. The object 10 can for example have been moved, so a new assignment is necessary. To this end the central processing unit 13 can for example, depending on a movement signal received via the connecting line 41, start a new detection of the object 10 to create a 3D dataset, or depending on a similarity parameter, and especially by means of the image processing unit 24, create a new 2D dataset from the 3D dataset which represents a view through the object 10, a view onto the object or a section through the object 10.

FIG. 2 shows a schematic diagram of an exemplary embodiment for a C-arm 84—which for example instead of the C-arm 9 shown in FIG. 1—can be part of the detection device 1. The C-arm 84 is connected at least indirectly to a control facility 86. The C-arm 84 features an x-ray source 82 and a detector 80. The x-ray source 82 is arranged in the area of a first end of the C-arm 84 and the detector 80 is arranged in the area of a second end of the C-arm 84 such that an object arranged in the area of a isocenter 65—for example the object 10 shown in FIG. 1—can be irradiated by means an x-ray emitted by the x-ray source 82 along a direction of detection 66.

The detector 80 is arranged and aligned so as to receive the x-ray sent out by the x-ray source 82. The C-arm 84 is embodied, guided by the control facility 86, to execute a translation movement along a longitudinal axis Y, along a transverse axis X, or along a vertical axis Z, or along a combination of these axes of translation.

The C-arm 9 is also embodied, guided by the control facility 86, to execute a pivot movement along a rotational degree Of freedom 67, along of a rotational degree of freedom 69 or along of a rotational degree of freedom 71. A rotational movement of the C-arm 84 in the rotational degree of freedom 67 or in the rotational degree of freedom 69 occurs in this case around an axis of rotation, which runs through the isocenter 65.

FIG. 3 shows an exemplary embodiment for a method for detecting an object by means of x-rays in up to three dimensions.

In a step 73 position datasets are kept stored which each represent different detection positions in relation to one another for creating a 2D dataset or a sequence of 2D datasets.

In a step 75 a plurality of 2D datasets is created by means of a detector for the x-rays, with the 2D datasets representing the object in directions of detection which differ from one another in each case in a projection through the object, and a 3D dataset representing the object in three spatial dimensions is created from the 2D datasets which represents the object in three dimensions.

In a step 77, for each position dataset the object is detected depending on a user interaction signal corresponding to the positions represented by the position datasets and the 2D dataset or the chronological sequence of 2D datasets are created for each detection position.

In a step 79 an image dataset is created from the 3D dataset, which represents the object, especially a view onto the object, a view through object or a section through the object, and this together with at least one 2D dataset or chronological sequence of 2D datasets representing the object in a projection through the object is reproduced by means of an image display unit.

1. A detection device (1) for detecting an object (10) in up to three dimensions,
with an x-ray source (3) which is embodied to emit x-rays (12) and a detector (5) arranged in a detection plane for the x-rays (12) which is arranged and embodied so as to detect the x-rays (12) and to create at least one 2D dataset which represents the object (10) in a projection through the object (10) onto the detection plane, with the detection device (1) featuring a C-arm (9) which is connected to the x-ray source (3) and to the detector (5).
and the detection device(1) is embodied to create a 3D dataset (19) representing the object (10) in three spatial dimensions from a plurality of 2D datasets (18) which represent the object (10) in different directions of detection in each case in a projection through the object (10) and to keep the 3D dataset (19) stored in a memory (17), characterized in that
the detection device (1) features a control facility (11) effectively connected to the C-arm (9) which is embodied to trove the C-arm (9) depending on a control signal received on the input side, in at least two or three rotational degrees of freedom and to hold it in a detection position represented by the control signal,
and the detection device (1) is embodied to create from the 3D dataset (19) an image dataset which represents the object, especially a view onto the object, a view through the object or a section through the object, and to reproduce this together with the at least one 2D dataset by means of at least one image display unit (26),
and the detection device (1) features a position memory (25) for position datasets (23) which each represent a detection position and the detection device (1) is embodied, depending on a user interaction signal, to read out at least one position dataset (23) from the position memory (25) and to create a corresponding control signal for moving the C-arm (9) into the detection position and in the detection position to create a 2D dataset (18) by means of the detector (5).

2. The detection device as claimed in claim 1, characterized in that
the detection device (1) features a location sensor (28) which is embodied to detect a location of a medical instrument (30, 30′) in a spatial area provided for the detection of the object (10), and to create a dataset representing the instrument location and to assign this to an area of the 3D dataset (19) corresponding to the instrument location and to create the image dataset depending on the instrument dataset such that the image dataset additionally represents the medical instrument (30′).

3. The detection device as claimed in one of the previous claims, characterized in that
the detection device features a coordinate memory (20) and is embodied to create an object coordinate dataset (22) representing at least one detection location of the 3D dataset (19) and to store the object coordinate dataset (22) in the coordinate memory (20), and to read out the object coordinate dataset (22) stored in the coordinate memory (20) and output the instrument location in relation to the read-out object coordinate dataset (22).

4. The detection device as claimed in one of the previous claims, characterized in that
the detection device (1) features an image processing unit (24) which is embodied to create from the 3D dataset (19) a 2D dataset which represents a projection through the object represented by the 3D dataset and to output this on the output side.

5. The detection device as claimed in one of the previous claims, characterized in that
the detection device is embodied by means of the detector (5) to create a chronological sequence of 2D datasets, each of which represents chronologically consecutive detection results of the object (10).

6. The detection device as claimed in one of the previous claims, characterized in that
the image processing unit (24) is embodied to subtract at least two 2D datasets for corresponding detection locations from each other and to create an angio 2D dataset representing the subtraction result (27).

7. The detection device as claimed in one of the previous claims, characterized in that
the detection device features a movement sensor (16) which is embodied to detect a movement of the object (10) and to create a movement signal which represents the object movement, and the detection device is embodied, depending on the movement signal, to create a 3D dataset (19) or to create a 2D dataset from the 3D dataset (19).

8. The detection device as claimed in one of the previous claims, characterized in that
the control facility (11) is embodied to move the C-arm (9) depending on the control signal in at least two or three translational degrees of freedom and to hold it in a detection position represented by the control signal.

9. A method for detecting an object (10) in up to three dimensions by means of x-rays (12), in which a plurality of 2D datasets are created by means of a detector (5) for the x-rays (12), with the 2D datasets each representing the object in different directions of detection in relation to each other in a projection through the object, and a 3D dataset (19) representing the object in three spatial dimensions is created from the 2D datasets which represents the object in three dimensions,
and an image dataset is created from the 3D dataset (19) which represents the object (10), especially a view onto the object, a view through the object or a section through the object, and this is reproduced together with at least one 2D dataset (18) or a chronological sequence of 2D datasets (18) created by the detector (5) representing the object (10) in a projection through the object by means of an image display unit (26), characterized in that
at least two position datasets (23) are kept stored which each represent different detection positions for creating a 2D dataset (18) and a detection of the object is undertaken for the position datasets (23) depending on a user interaction signal corresponding to the detection position represented by the position datasets in each case and the 2D dataset (18) or the chronological sequence of 2D datasets (18) is created in the detection position by means of the detector.

10. The method as claimed in claim 9, characterized in that
an instrument location of a medical instrument (30) is detected in a spatial area provided for the detection of the object (10) and an instrument dataset representing the instrument location is created and assigned to an area of the 3D dataset (19) corresponding to the instrument location,
and an image dataset is created from the 3D dataset (19) which represents the object (10), especially a view onto the object, a view through the object or a section through the object together with the instrument (30).

11. A method as claimed in one of claims 9 or 10, in which a subtraction result is created from at least two 2D datasets (18) by subtraction for corresponding detection locations and an angio 2D dataset (27) representing this is created and this is reproduced, together with the image data, by means of the image display unit (26).
